(19) United States
(12) Patent Application Publication
(10) Pub. No.: US 2019/0183327 A1
(43) Pub. Date: Jun. 20, 2019

(54) ENDOSCOPE REPROCESSOR

(71) Applicant: OLYMPUS CORPORATION, Tokyo (JP)

(72) Inventor: Koichiro OKADA, Tokyo (JP)

(73) Assignee: OLYMPUS CORPORATION, Tokyo (JP)

(21) Appl. No.: 16/284,000

(22) Filed: Feb. 25, 2019

Related U.S. Application Data
(63) Continuation of application No. PCT/JP2017/011251, filed on Mar. 21, 2017.

(30) Foreign Application Priority Data
Sep. 13, 2016 (JP) 2016-178641

(51) Int. Cl. A61B 1/12; A61L 2/18

(52) U.S. Cl. CPC A61B 1/125; A61L 2202/24; A61B 1/123; A61L 2/18

(57) ABSTRACT

An endoscope reprocessor includes: a treatment tank in which an endoscope is arranged; a drainage conduit including a liquid lead-out port opened in a bottom surface of the treatment tank; an on-off valve arranged in an intermediate position of the drainage conduit; a circulation conduit including a circulation port, the circulation port being opened between the on-off valve and the liquid lead-out port of the drainage conduit and in a position closer to a ground than the liquid lead-out port, the circulation conduit introducing liquid introduced from the circulation port to the treatment tank; a filter arranged in the drainage conduit and covering the circulation port; and a holding portion arranged in the filter or the drainage conduit, the holding portion holding the filter at a position at which the circulation port is covered.

### CROSS REFERENCE TO RELATED APPLICATION

This application is a continuation application of PCT/JP2017/011251 filed on Mar. 21, 2017 and claims benefit of Japanese Application No. 2016-178641 filed in Japan on Sep. 13, 2016, the entire contents of which are incorporated herein by this reference.

### BACKGROUND OF THE INVENTION

### 1. Field of the Invention

The present invention relates to an endoscope reprocessor including a filter arranged in a flow path of liquid.

### 2. Description of the Related Art

Reprocessing using a medicinal solution such as an antiseptic solution after use is applied to endoscopes used in the medical field. Further, as disclosed in Japanese Patent Application Laid-Open Publication No. 2010-57752, an endoscope cleaning/disinfecting apparatus that automatically performs reprocessing by arranging an endoscope in a cleaning tank and introducing liquid such as a cleaning solution and a medicinal solution in the cleaning tank is known, for example.

The endoscope cleaning/disinfecting apparatus disclosed in Japanese Patent Application Laid-Open Publication No. 2010-57752 includes an opening for leading out the liquid accumulated in the cleaning tank, and a circuit for introducing the liquid led out from the opening into a conduit of the endoscope.

Further, a feature in which a filter is arranged on an opening part of the cleaning tank in the endoscope cleaning/disinfecting apparatus so that foreign matters existing in the liquid do not enter the conduit of the endoscope and the foreign matters are collected by the filter is known.

### SUMMARY OF THE INVENTION

An endoscope reprocessor according to one aspect of the present invention includes: a treatment tank in which an endoscope is arranged; a drainage conduit including a liquid lead-out port opened in a bottom surface of the treatment tank; an on-off valve arranged in an intermediate position of the drainage conduit; a circulation conduit including a circulation port, the circulation port being opened between the on-off valve and the liquid lead-out port of the drainage conduit and in a position closer to a ground than the liquid lead-out port, the circulation conduit introducing liquid introduced from the circulation port to the treatment tank; a filter arranged in the drainage conduit and covering the circulation port; and a holding portion arranged in the filter or the drainage conduit, the holding portion holding the filter at a position at which the circulation port is covered and liquid that flows into the drainage conduit from an inside of the treatment tank through the circulation port passes through the filter and liquid that flows toward the on-off valve from the inside of the treatment tank through the liquid lead-out port flows along a surface of the filter when the on-off valve is in an open state.

### BRIEF DESCRIPTION OF THE DRAWINGS

FIG. 1 is a view illustrating a configuration of an endoscope reprocessor of a first embodiment;

FIG. 2 is a view illustrating a liquid lead-out port, a vertical conduit portion, a circulation port, a filter, and a holding portion of the endoscope reprocessor of the first embodiment in an enlarged manner;

FIG. 3 is a schematic diagram illustrating a flow of liquid when the liquid is circulated so as to pass through a treatment tank and a circulation conduit in the endoscope reprocessor of the first embodiment;

FIG. 4 is a schematic diagram illustrating a flow of liquid when the liquid in the treatment tank is drained via a drainage conduit in the endoscope reprocessor of the first embodiment;

FIG. 5 is a view illustrating a first modification of the first embodiment;

FIG. 6 is a view illustrating a second modification of the first embodiment;

FIG. 7 is a view illustrating a third modification of the first embodiment;

FIG. 8 is a view illustrating a fourth modification of the first embodiment;

FIG. 9 is a view illustrating a liquid lead-out port, a vertical conduit portion, a circulation port, a filter, and a holding portion in an endoscope reprocessor of a second embodiment in an enlarged manner;

FIG. 10 is a perspective view illustrating the filter and a filter holding cylinder of the second embodiment;

FIG. 11 is a cross-sectional view of FIG. 9 taken along line XI-XI;

FIG. 12 is a schematic diagram illustrating a flow of liquid when the liquid is circulated so as to pass through a treatment tank and a circulation conduit in the endoscope reprocessor of the second embodiment;

FIG. 13 is a schematic diagram illustrating a flow of liquid when the liquid in the treatment tank is drained via the drainage conduit in the endoscope reprocessor of the second embodiment;

FIG. 14 is a view illustrating a first modification of the second embodiment;

FIG. 15 is a view illustrating a second modification of the second embodiment;

FIG. 16 is a view illustrating a third modification of the second embodiment; and

FIG. 17 is a view illustrating a liquid lead-out port, a vertical conduit portion, a circulation port, a filter, and a holding portion of an endoscope reprocessor of a third embodiment in an enlarged manner.

### DETAILED DESCRIPTION OF THE PREFERRED EMBODIMENT(S)

Preferred embodiments of the present invention are described below with reference to the drawings. Note that, in each of the drawings referred to in the description below, the scale is different for each of the components so that each of the components is represented in a size that is recognizable in the drawings. Further, the present invention is not limited only to the number of the components, shapes of the components, size ratios of the components, and relative positional relationships among the respective components illustrated in the drawings.

### First Embodiment

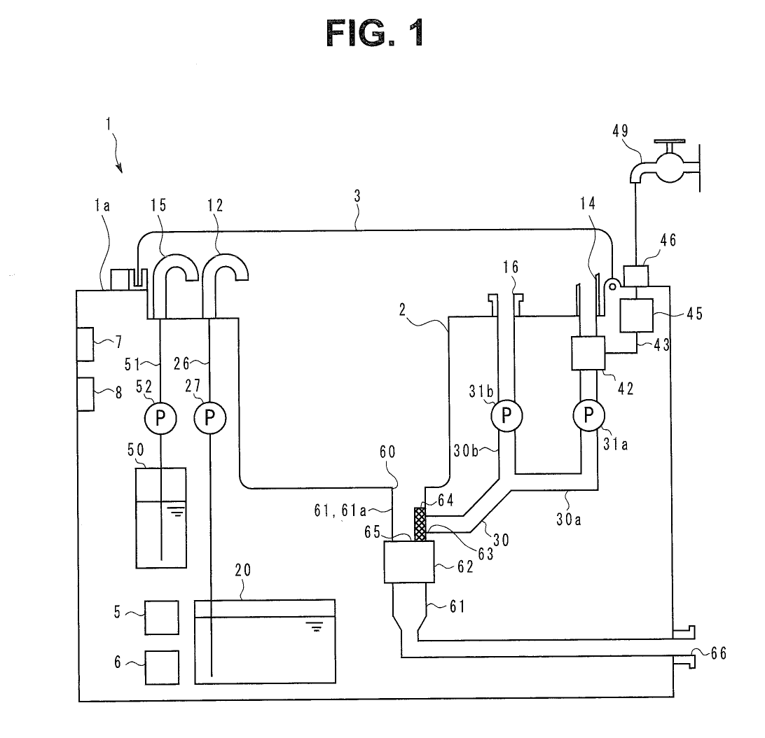

An example of an embodiment of the present invention is described below. An endoscope reprocessor 1 illustrated in FIG. 1 is an apparatus that applies reprocessing to an endoscope. The reprocessing herein is not particularly limited, and may be any one of rinsing process performed by water, cleaning process for removing contaminations such as organic matters, disinfection process for disabling specified microorganisms, and sterilization process for eliminating or sterilizing all microorganisms, or a combination of the abovementioned processes.

Note that, in the description below, expressions “above”, “upper side”, and “upward” refer to a position farther from a ground with respect to an object to be compared and expressions “below”, “lower side”, and “downward” refer to a position closer to the ground with respect to the object to be compared. Further, expressions “high” and “low” in the description below indicate a height relationship along a direction of gravity.

The endoscope reprocessor 1 includes a control unit 5, a power supply unit 6, a treatment tank 2, a liquid lead-out port 60, a drainage conduit 61, an on-off valve 62, a circulation conduit 30, a circulation port 63, a filter 64, and a holding portion 65.

The control unit 5 can be configured to include a computing unit (CPU), a storage device (RAM), an auxiliary storage device, an input-output device, a power control device, and the like, and has a configuration that executes a predetermined program in accordance with instructions from a user and controls operation of each part configuring the endoscope reprocessor 1. The operation of each configuration included in the endoscope reprocessor 1 in the description below is controlled by the control unit 5 even when there are no descriptions in particular.

An operation portion 7 and a display unit 8 configure a user interface that performs the exchange of information between the control unit 5 and the user. The operation portion 7 includes operation members that receive operation instructions from the user such as a push-button switch and a touch sensor. The operation instructions from the user are converted to electrical signals by the operation portion 7 and are inputted to the control unit 5. The operation instructions from the user are instructions for starting the reprocessing, for example. Note that a faun configured so that the operation portion 7 is included in an electronic device separate from a main body portion 1a of the endoscope reprocessor 1 and the electronic device performs wired communication or wireless communication with the control unit 5 may be employed.

Further, the display unit 8 includes, for example, a display device that displays images and characters, a light-emitting device that emits light, a speaker that emits sound, a vibrator that generates vibration, or combinations of the above. The display unit 8 outputs information from the control unit 5 to the user. Note that a form configured so that the display unit 8 is included in an electronic device separate from the main body portion 1a of the endoscope reprocessor 1 and the electronic device performs wired communication or wireless communication with the control unit 5 may be employed.

The power supply unit 6 supplies electric power to each part of the endoscope reprocessor 1. The power supply unit 6 distributes electric power acquired from outside such as a commercial power supply to each part. Note that the power supply unit 6 may include an electric generator and a battery.

The treatment tank 2 has a recessed shape having an opening portion, and liquid can be accumulated in the treatment tank 2. An endoscope (not shown) can be arranged in the treatment tank 2. A plurality of endoscopes may be arrangeable in the treatment tank 2.

A lid 3 for opening and closing the opening portion of the treatment tank 2 is provided on an upper portion of the treatment tank 2. When the reprocessing is applied to the endoscope in the treatment tank 2, the opening portion of the treatment tank 2 is closed by the lid 3.

A medicinal solution nozzle 12, a cleaning solution nozzle 15, a liquid lead-out port 60, a circulation nozzle 14, and an endoscope connecting portion 16 are provided in the treatment tank 2.

The medicinal solution nozzle 12 is an opening portion communicating with a medicinal solution tank 20 via a medicinal solution conduit 26. A medicinal solution is accumulated in the medicinal solution tank 20.

The type of the medicinal solution accumulated in the medicinal solution tank 20 is not particularly limited, but the medicinal solution is an antiseptic solution used in the disinfection process or a sterilizing solution used in the sterilization process in the present embodiment as an example. The antiseptic solution or the sterilizing solution includes a peracetic acid aqueous solution.

A medicinal solution pump 27 is provided in the medicinal solution conduit 26. By operating the medicinal solution pump 27, the medicinal solution in the medicinal solution tank 20 is transferred into the treatment tank 2 via the medicinal solution conduit 26 and the medicinal solution nozzle 12.

Further, in the present embodiment, as an example, the medicinal solution is reusable when the medicinal solution has medical efficacy after being used in the reprocessing. The endoscope reprocessor 1 has a configuration that collects the medicinal solution in the treatment tank 2 and returns the medicinal solution into the medicinal solution tank 20, although the configuration is not shown.

The cleaning solution nozzle 15 is an opening portion communicating with the cleaning solution tank 50 in which the cleaning solution is accumulated via the cleaning solution conduit 51. The cleaning solution is used in the cleaning process. A cleaning solution pump 52 is provided in the cleaning solution conduit 51. The cleaning solution pump 52 is electrically connected to the control unit 5, and the operation of the cleaning solution pump 52 is controlled by the control unit 5. By operating the cleaning solution pump 52, the cleaning solution in the cleaning solution tank 50 is transferred into the treatment tank 2.

The liquid lead-out port 60 is an opening portion provided in a lowest location in the treatment tank 2. The liquid lead-out port 60 is provided in a location in which the liquid in the treatment tank 2 is gathered by gravity. In the present embodiment illustrated in FIG. 1, as an example, the liquid lead-out port 60 is opened in a bottom surface of the treatment tank 2. Note that the liquid lead-out port 60 may be opened in a corner portion between the bottom surface and a side surface.

The drainage conduit 61 is connected to the liquid lead-out port 60. In other words, the drainage conduit 61 has the liquid lead-out port 60 opening to the inside of the treatment tank 2.

The drainage conduit 61 communicates with the liquid lead-out port 60 and a discharge port 66. The discharge port 66 is a part that discharges the liquid to outside used by the endoscope reprocessor 1. The discharge port 66 is connected to a drainage appliance for receiving the liquid discharged from the endoscope reprocessor, for example.

The on-off valve 62 is arranged in an intermediate position of the drainage conduit 61. The on-off valve 62 can be at least switched between a closed state in which the drainage conduit 61 is closed and an open state in which the drainage conduit 61 is opened. The on-off valve 62 is electrically connected to the control unit 5, and the operation of the on-off valve 62 is controlled by the control unit 5. When the on-off valve 62 is in the closed state, liquid can be accumulated in the treatment tank 2. Further, when the on-off valve 62 is in the open state, the liquid in the treatment tank 2 is discharged from the discharge port 66 via the liquid lead-out port 60 and the drainage conduit 61. Note that a pump for facilitating the discharge of the liquid from the treatment tank 2 may be provided between the on-off valve 62 and the discharge port 66 of the drainage conduit 61.

The circulation port 63 is opened between the on-off valve 62 and the liquid lead-out port 60 of the drainage conduit 61 and in a position (low position) closer to the ground than the liquid lead-out port. The circulation conduit 30 is connected to the circulation port 63. In other words, the circulation conduit 30 includes the circulation port 63. The circulation conduit 30 is a conduit that introduces the liquid introduced from the circulation port 63 to the treatment tank 2 by the configuration described below. The circulation conduit 30 has a section arranged in a position (high position) farther from the ground than the circulation port 63.

In the present embodiment, as an example, the circulation conduit 30 branches into two conduits, that is, a treatment tank circulation conduit 30a and an endoscope connecting conduit 30b.

The treatment tank circulation conduit 30a is connected to the circulation nozzle 14. The circulation nozzle 14 is an opening portion provided in the treatment tank 2. The circulation nozzle 14 is opened in a position farther from the ground than the liquid lead-out port 60. Therefore, the treatment tank circulation conduit 30a has a section arranged in a position farther from the ground than the circulation port 63.

A treatment tank circulation pump 31a is provided in the treatment tank circulation conduit 30a. The treatment tank circulation pump 31a is electrically connected to the control unit 5, and the operation of the treatment tank circulation pump 31a is controlled by the control unit 5.

Further, a three-way valve 42 is provided between the treatment tank circulation pump 31a in the treatment tank circulation conduit 30a and the circulation nozzle 14. A water supply conduit 43 is connected to the three-way valve 42. The three-way valve 42 can be switched between a state in which the circulation nozzle 14 and the treatment tank circulation conduit 30a are communicating with each other or a state in which the circulation nozzle 14 and the water supply conduit 43 are communicating with each other.

The water supply conduit 43 causes the three-way valve 42 and a water supply source connecting portion 46 to communicate with each other. A water introduction valve 45 that opens and closes the water supply conduit 43 and a water filter 44 that filters water are provided in the water supply conduit 43. The water supply source connecting portion 46 is connected to a water supply source 49 such as waterworks that transmits water, for example, via a hose and the like. The three-way valve 42 and the water introduction valve 45 are electrically connected to the control unit 5, and the operation of the three-way valve 42 and the water introduction valve 45 is controlled by the control unit 5.

When liquid is accumulated in the treatment tank 2, when the operation of the treatment tank circulation pump 31a is started while the three-way valve 42 is in a state in which the circulation nozzle 14 and the treatment tank circulation conduit 30a are communicating with each other, the liquid in the treatment tank 2 is introduced to the circulation conduit 30 (treatment tank circulation conduit 30a) from the liquid lead-out port 60, and then ejected from the circulation nozzle 14, to thereby be introduced into the treatment tank 2.

Further, when the water introduction valve 45 is caused to be in the open state while the three-way valve 42 is in a state in which the circulation nozzle 14 and the water supply conduit 43 are communicating with each other, water supplied from the water supply source 49 is introduced into the treatment tank 2 via the circulation nozzle 14.

The endoscope connecting conduit 30b is connected to the endoscope connecting portion 16. The endoscope connecting portion 16 is connected to a pipe sleeve provided in an endoscope arranged in the treatment tank 2. The endoscope connecting portion 16 may be in a form of being directly connected to the pipe sleeve, or may be in a form of being connected to the pipe sleeve via a connection tube. The endoscope connecting portion 16 is arranged in a position farther from the ground than the liquid lead-out port 60. Therefore, the endoscope connecting conduit 30b has a section arranged in a position farther from the ground than the circulation port 63.

An endoscope circulation pump 31b is provided in the endoscope connecting conduit 30b. The endoscope circulation pump 31b is electrically connected to the control unit 5, and the operation of the endoscope circulation pump 31b is controlled by the control unit 5.

When the operation of the endoscope circulation pump 31b is started when liquid is accumulated in the treatment tank 2, the liquid in the treatment tank 2 is introduced to the circulation conduit 30 (endoscope connecting conduit 30b) from the liquid lead-out port 60, and then introduced into the treatment tank 2 via a conduit that communicates with the endoscope connecting portion 16 and the pipe sleeve of the endoscope.

As described above, the endoscope reprocessor 1 of the present embodiment has a configuration that introduces the liquid introduced from the liquid lead-out port 60 to the circulation conduit 30 into the treatment tank 2.

The filter 64 is arranged between the on-off valve 62 and the liquid lead-out port 60 of the drainage conduit 61, and covers the circulation port 63. The holding portion 65 is arranged in the filter 64 or the drainage conduit 61, and holds the filter 64 in a position that covers the circulation port 63.

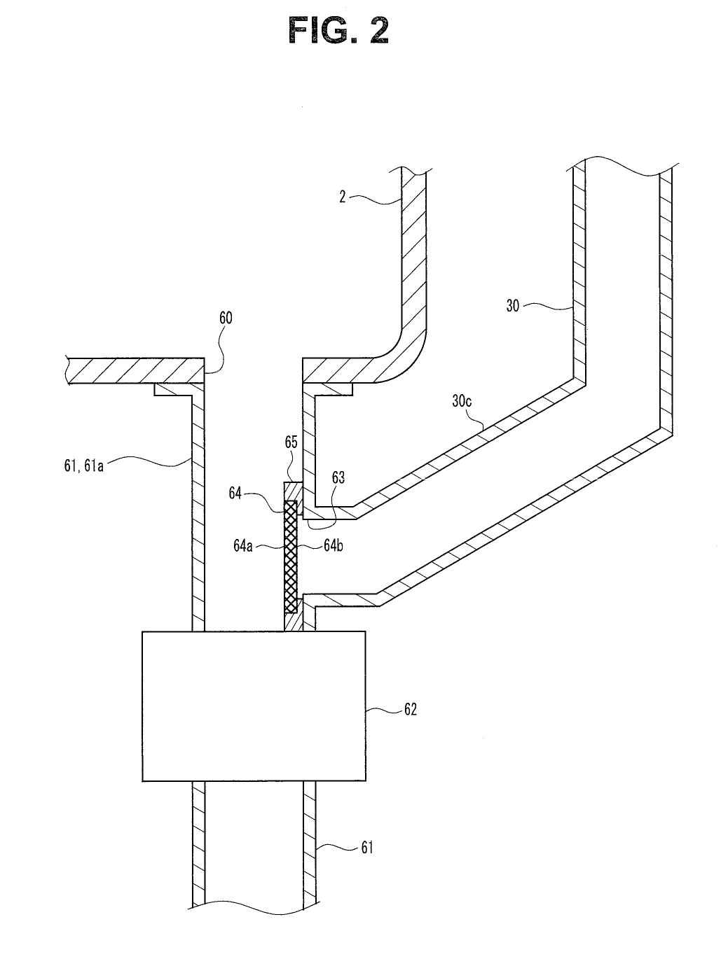

Next, detailed configurations of the liquid lead-out port 60, the drainage conduit 61, the circulation port 63, the circulation conduit 30, the filter 64, and the holding portion 65 are described. FIG. 2 is a view illustrating the liquid lead-out port 60, the drainage conduit 61, the circulation port 63, the filter 64, and the holding portion 65 in an enlarged manner.

For the filter 64 covering the circulation port 63, a surface exposed in the drainage conduit 61 is referred to as a first surface 64a and a surface exposed in the circulation conduit 30 is referred to as a second surface 64b.

As illustrated in FIG. 2, the drainage conduit 61 includes a vertical conduit portion 61a vertically arranged at least from the liquid lead-out port 60 to the circulation port 63. Therefore, the circulation port 63 is opened in a vertical wall surface of the drainage conduit 61. Further, the first surface 64a of the filter 64 covering the circulation port 63 is vertically arranged in the drainage conduit 61.

Note that the liquid lead-out port 60 may be covered with a netlike member that prevents attachments and the like of the endoscope from falling into the vertical conduit portion 61a of the circulation conduit 30, although the member is not shown in FIG. 2. In the abovementioned case, the netlike member has sparser mesh than the filter 64.

The circulation conduit 30 has an inclined conduit portion 30c inclined in a direction away from the ground when the circulation port 63 is a base point. That is, the circulation conduit 30 has a section arranged in a position farther from the ground than the circulation port 63 in the inclined conduit portion 30c.

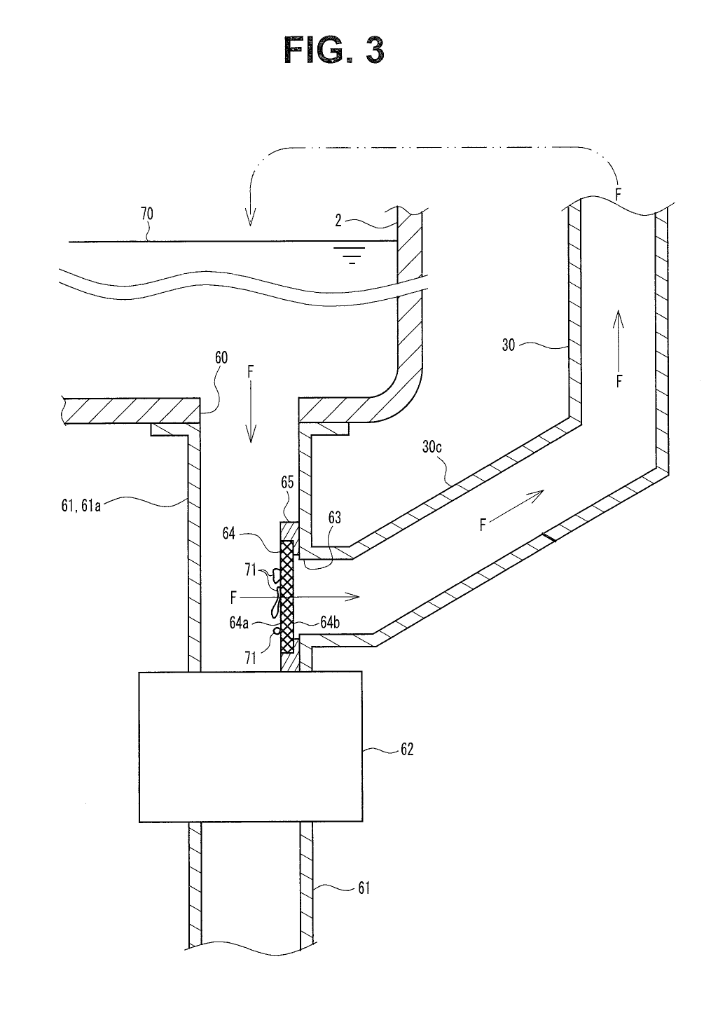

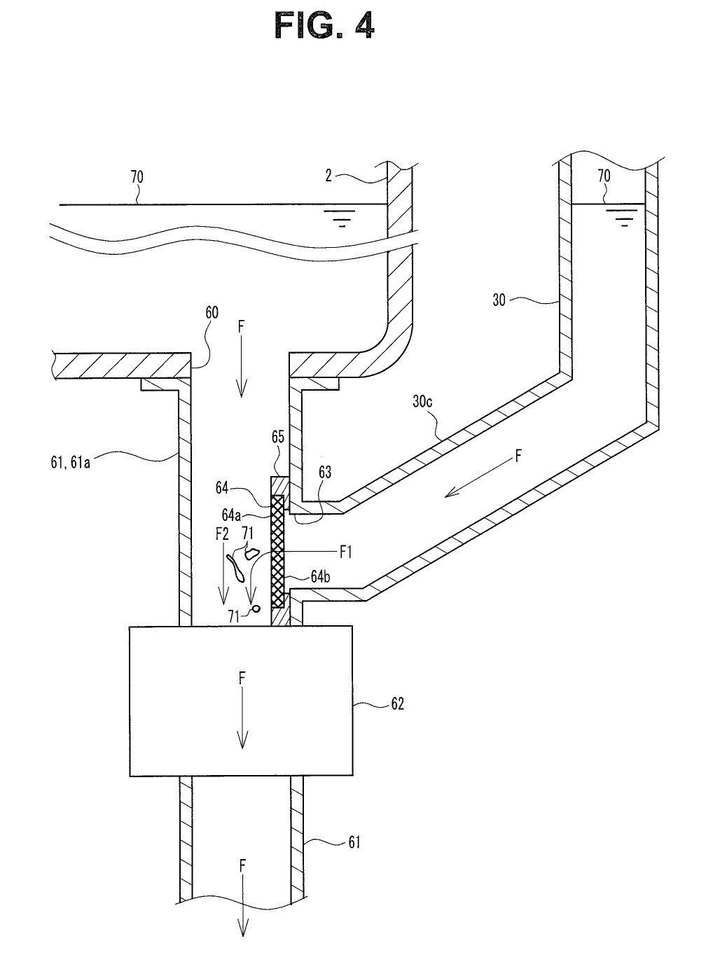

Next, an effect of the endoscope reprocessor 1 of the present embodiment having the configuration described above is described. FIG. 3 is a schematic diagram illustrating a flow of liquid when the liquid is circulated so as to pass through the treatment tank 2 and the circulation conduit 30. Further, FIG. 4 is a schematic diagram illustrating a flow of liquid when the liquid in the treatment tank 2 is drained via the drainage conduit 61.

The endoscope reprocessor 1 executes the reprocessing using liquid such as a medicinal solution, a cleaning solution, and water for the endoscope arranged in the treatment tank 2 by introducing the liquid into the treatment tank 2.

For example, when the cleaning process of the endoscope is executed, the control unit 5 of the endoscope reprocessor 1 causes the on-off valve 62 to be in the closed state, and then controls the cleaning solution pump 52 and the water introduction valve 45, to thereby introduce the cleaning solution and the water into the treatment tank 2 by a predetermined volume ratio and accumulate a liquid mixture of the cleaning solution and the water in the treatment tank 2.

Further, the control unit 5 controls the operation of the treatment tank circulation pump 31a and the endoscope circulation pump 31b, to thereby cause the liquid mixture to circulate so as to pass through the treatment tank 2 and the circulation conduit 30. By the circulation of the liquid mixture, an outer surface of the endoscope and the inside of the conduit are cleaned by the flowing liquid mixture.

Further, the control unit 5 stops the circulation of the liquid mixture, and then drains the liquid mixture in the treatment tank 2 via the drainage conduit 61 by causing the on-off valve 62 to be in the open state.

When the disinfection process or the sterilization process using a medicinal solution is executed or when the rinsing process using water is executed, the control unit 5 similarly introduces a predetermined volume of the medicinal solution or water into the treatment tank 2, and then causes the medicinal solution or the water to circulate so as to pass through the treatment tank 2 and the circulation conduit 30. Then, the medicinal solution or the water in the treatment tank 2 is drained via the drainage conduit 61.

As described above, when the reprocessing is performed, the endoscope reprocessor 1 of the present embodiment executes circulation operation that causes liquid to circulate so as to pass through the treatment tank 2 and the circulation conduit 30, and then executes drainage operation that drains the liquid in the treatment tank 2 via the drainage conduit 61.

FIG. 3 indicates a flow of liquid when the circulation operation is being executed by arrows F. As illustrated in FIG. 3, during the circulation operation, liquid 70 accumulated in the treatment tank 2 flows so as to flow into the vertical conduit portion 61a of the drainage conduit 61 from the liquid lead-out port 60, passes through the filter 64, and then returns into the treatment tank 2 through the circulation port 63 and the circulation conduit 30.

During the circulation operation, the liquid passes through the filter 64 from the first surface 64a toward the second surface 64b. Therefore, a foreign matter 71 detached from the endoscope and floating in the liquid 70 is collected by the filter 64 and adheres to the first surface 64a of the filter 64. The foreign matter 71 is biomedical tissue, mucus, and the like that has adhered to the endoscope when the endoscope is used, for example.

As described above, by arranging the filter 64 in a flow path of the liquid 70 during the circulation operation, the foreign matter 71 existing in the liquid 70 is collected, and the foreign matter is prevented from entering the circulation conduit 30 in which the pump is arranged or the conduit of the endoscope.

FIG. 4 indicates a flow of liquid during the execution of the drainage operation by arrows F. A state in which the liquid is accumulated in the treatment tank 2 is obtained at a time point immediately before the drainage operation starts. As described above, the drainage operation is executed after the circulation operation. Further, the circulation conduit 30 is communicating with the liquid lead-out port 60 of the treatment tank 2 via the vertical conduit portion 61a and the circulation port 63. Therefore, at a time point immediately before the drainage operation starts, a state in which the liquid 70 is accumulated not only in the treatment tank 2 but also in the circulation conduit 30 is obtained as illustrated in FIG. 4.

Further, when the drainage operation starts by causing the on-off valve 62 to be in the open state, the liquid 70 accumulated in the treatment tank 2 is drained through the drainage conduit 61 and the on-off valve 62. Further, the circulation conduit 30 has a section arranged in a position farther from the ground than the circulation port 63 such as the inclined conduit portion 30c. Therefore, when the drainage operation starts, at least a part of the liquid 70 accumulated in the circulation conduit 30 flows toward the circulation port 63, and is drained through the circulation port 63, the drainage conduit 61, and the on-off valve 62.

Now, when the flow of the liquid 70 near the filter 64 during the drainage operation is focused on, during the drainage operation, a first flow F1 in which the liquid 70 passes through the filter 64 from the second surface 64b toward the first surface 64a from the inside of the circulation conduit 30 and then flows into the vertical conduit portion 61a, and a second flow F2 in which the liquid 70 flows into the vertical conduit portion 61a from the treatment tank 2 and then flows down to the lower side along the first surface 64a of the filter 64 are generated. The first flow F1 and the second flow F2 are indicated by an arrow F1 and an arrow F2 in FIG. 4.

The first flow F1 in the liquid 70 generated during the drainage operation applies force to the foreign matter 71 adhering to the first surface 64a of the filter 64 in a direction in which the foreign matter 71 is detached from the first surface 64a into the vertical conduit portion 61a. Therefore, by the first flow F1 in the liquid 70, the foreign matter 71 adhering to the first surface 64a of the filter 64 is placed in a state that is detached or easily detached from the first surface 64a.

Further, the second flow F2 in the liquid 70 generated during the drainage operation causes the foreign matter 71 adhering to the first surface 64a of the filter 64 to be detached from the first surface 64a by applying shear force to the foreign matter 71. Further, the second flow F2 in the liquid 70 washes away the foreign matter 71 detached from the first surface 64a to the on-off valve 62 and discharges the foreign matter 71 from the inside of the vertical conduit portion 61a.

As described above, the endoscope reprocessor 1 of the present embodiment detaches the foreign matter 71 collected by the filter 64 from the filter 64 and discharges the foreign matter 71 with the liquid 70 when the drainage operation of the liquid 70 accumulated in the treatment tank 2 is executed. That is, the endoscope reprocessor 1 of the present embodiment can automatically remove the foreign matter 71 from the filter 64. As a result, the frequency of cleaning work of the filter 64 manually performed by the user can be reduced, for example.

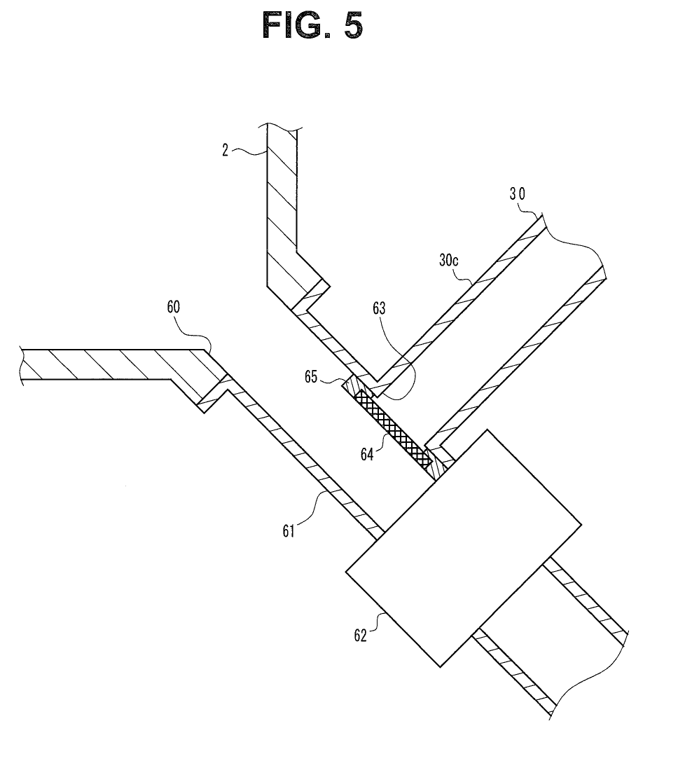

Note that, in the present embodiment, the section from the liquid lead-out port 60 to the circulation port 63 in the drainage conduit 61 is vertically arranged, but the arrangement of the section from the liquid lead-out port 60 to the circulation port 63 in the drainage conduit 61 is not limited to the present embodiment. For example, as in a first modification illustrated in FIG. 5, the section from the liquid lead-out port 60 to the circulation port 63 in the drainage conduit 61 may be arranged so as to be tilted with respect to the direction of gravity. Further, as in the first modification illustrated in FIG. 5, the liquid lead-out port 60 may be provided in a corner portion at which the bottom surface and the side surface of the treatment tank 2 intersect with each other.

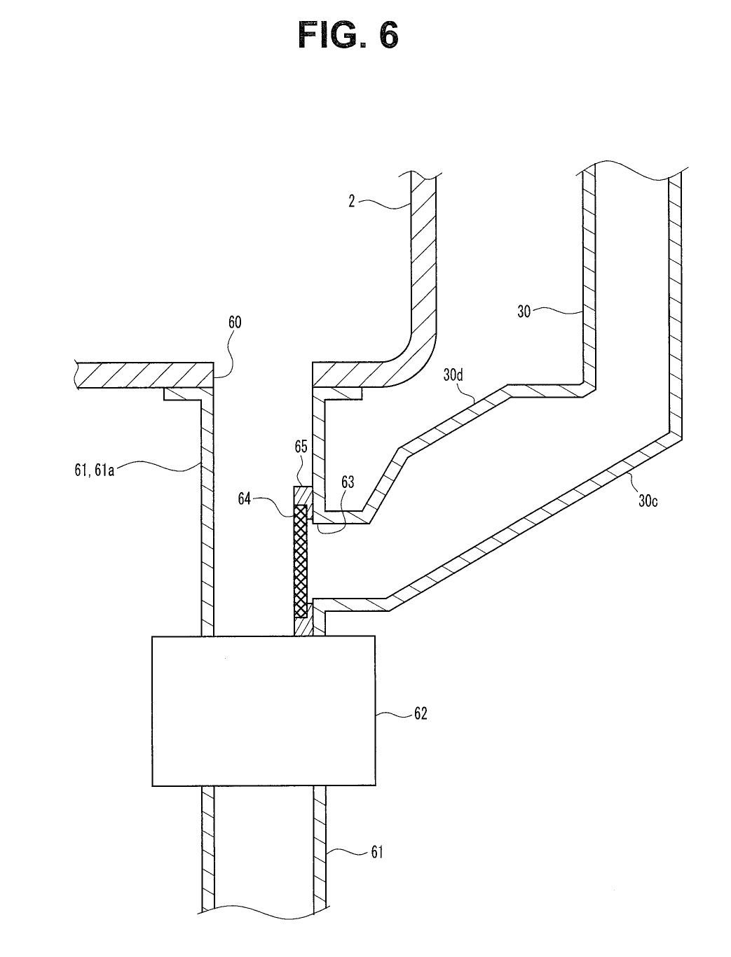

Further, a cross-sectional area of the flow path of the circulation conduit 30 does not necessarily need to be constant, and may be different for each predetermined section. For example, as in a second modification illustrated in FIG. 6, an enlarged diameter portion 30d having a cross-sectional area that is expanded as compared to cross-sectional areas of other sections of the circulation conduit 30 may be provided in the inclined conduit portion 30c of the circulation conduit 30. By providing the enlarged diameter portion 30d in the inclined conduit portion 30c of the circulation conduit 30, a volume of the liquid accumulated in the circulation conduit 30 at a time point in which the drainage operation starts can be increased. Therefore, in the second modification, the volume of the liquid flowing in the direction in which the foreign matter 71 is detached from the filter 64 in the drainage operation can be increased, and hence the foreign matter can be removed from the filter 64 in a more reliable manner.

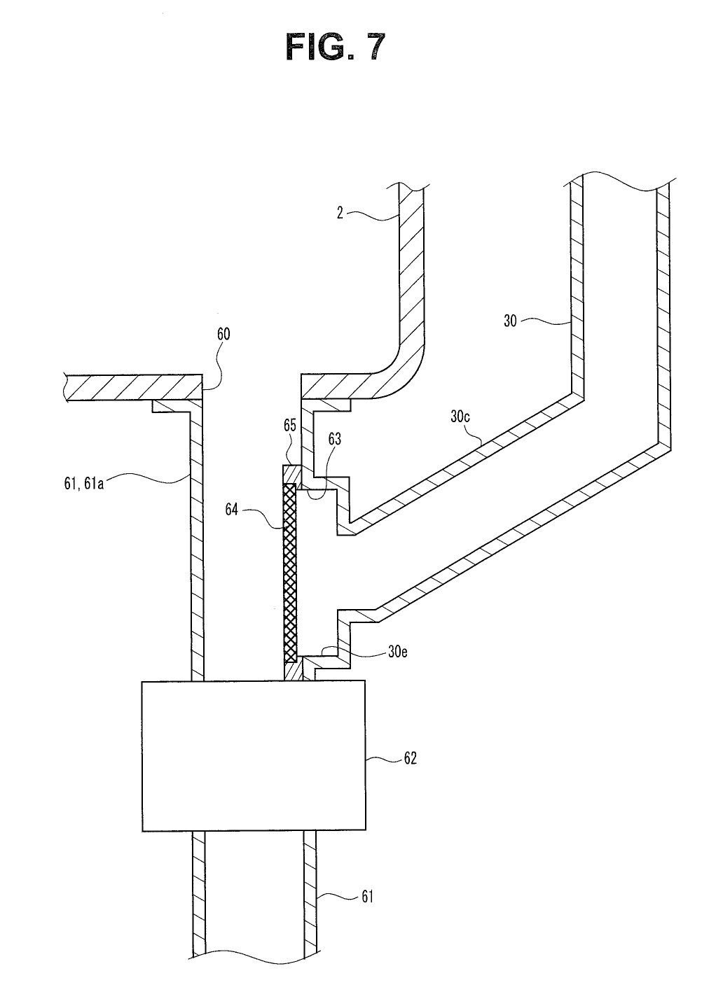

Further, as in a third modification illustrated in FIG. 7, an enlarged diameter portion 30e that expands a cross-sectional area of the flow path may be provided in a portion at which the circulation conduit 30 is connected to the circulation port 63. By providing the enlarged diameter portion 30e in the portion at which the circulation conduit 30 is connected to the circulation port 63, an area by which the liquid passes through the filter 64 during the circulation operation expands, and a flow rate of the liquid passing through the filter 64 can be increased.

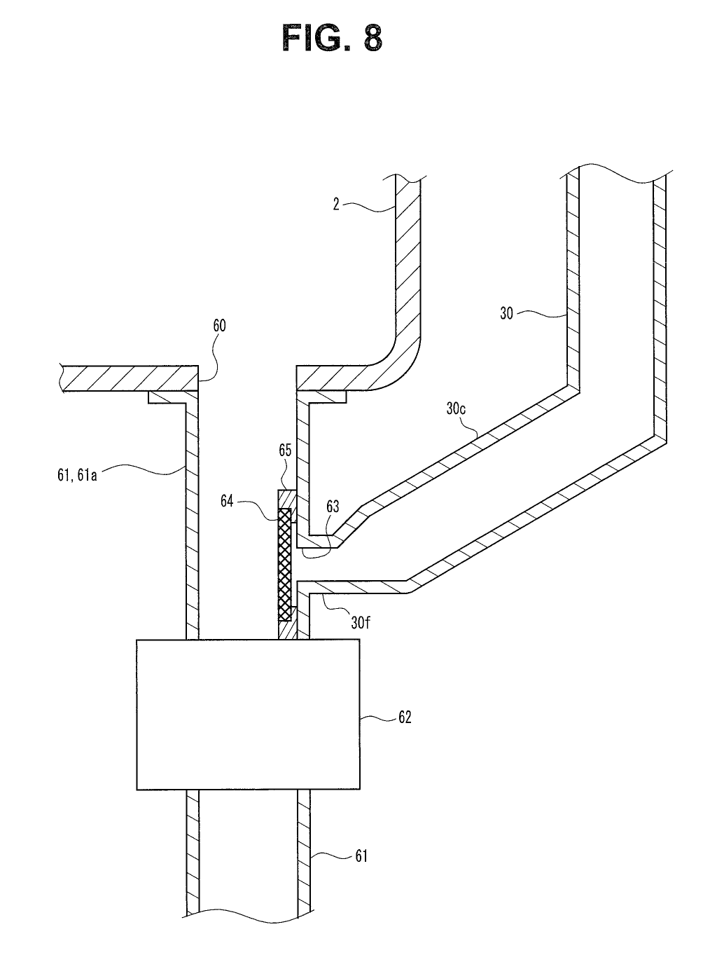

Further, as in a fourth modification illustrated in FIG. 8, a reduced diameter portion 30f that reduces the cross-sectional area of the flow path may be provided in a portion at which the circulation conduit 30 is connected to the circulation port 63. By providing the reduced diameter portion 30f in the portion at which the circulation conduit 30 is connected to the circulation port 63, a flow velocity of the liquid flowing in a direction in which the foreign matter 71 is detached from the filter 64 in the drainage operation can be increased, and hence the foreign matter can be removed from the filter 64 in a more reliable manner.

### Second Embodiment

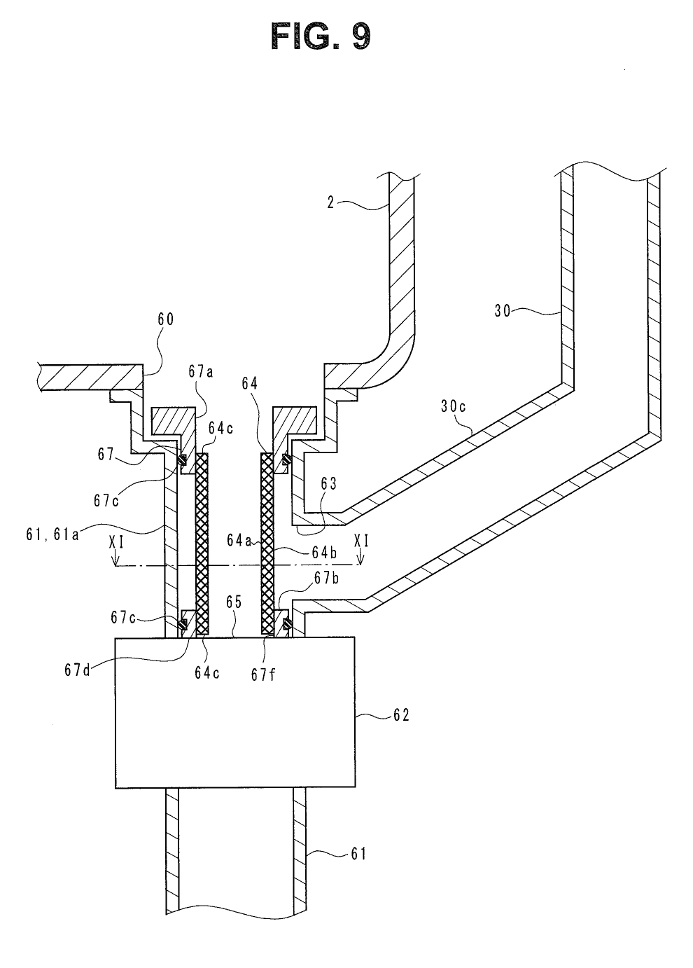

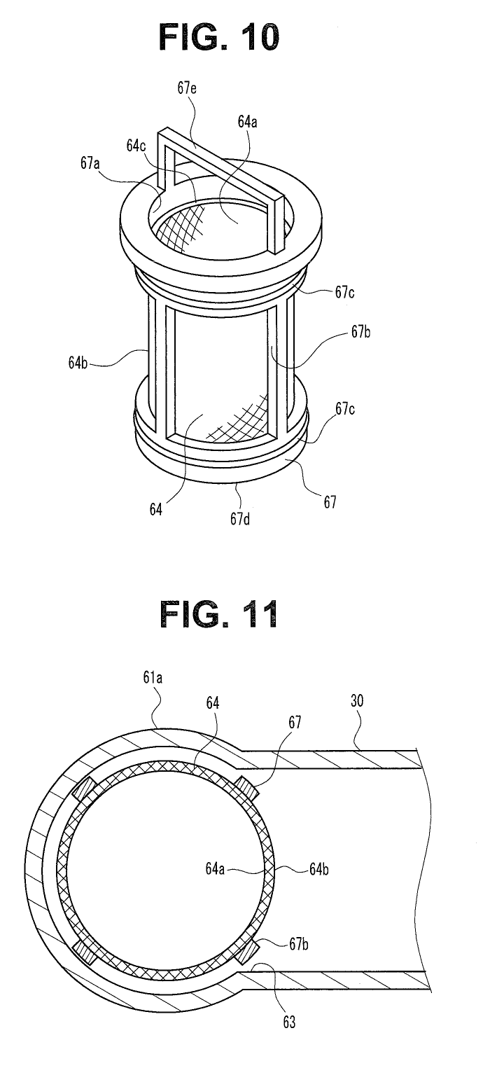

A second embodiment of the present invention is described below. Only differences from the first embodiment are described below, components similar to components in the first embodiment are denoted by the same reference characters, and description of such components is omitted as appropriate. FIG. 9 is a view illustrating the liquid lead-out port 60, the vertical conduit portion 61a, the circulation port 63, the filter 64, and the holding portion 65 of the endoscope reprocessor 1 of the present embodiment in an enlarged manner. FIG. 10 is a perspective view illustrating the filter 64 and a filter holding cylinder 67. FIG. 11 is a cross-sectional view of FIG. 9 taken along line XI-XI.

The present embodiment is different from the first embodiment in terms of the configuration of the filter 64 and the holding portion 65. The filter 64 of the present embodiment has a cylindrical shape, both ends of which are opened. In the filter 64, a pair of end portions 64c face upward and downward in a state in which the filter 64 is inserted from the liquid lead-out port 60 into the vertical conduit portion 61a. Therefore, when the filter 64 having a cylindrical shape is inserted in the vertical conduit portion 61a, the end portion 64c facing upward is opened toward the liquid lead-out port 60. Further, when the filter 64 is inserted in the vertical conduit portion 61a, the end portion 64c facing downward is opened toward the on-off valve 62. The filter 64 has a cylindrical shape, and hence the openings at the pair of end portions 64c are connected by a space passing through the filter 64. Therefore, in the drainage operation in which the on-off valve 62 is caused to be in the open state and the liquid in the treatment tank 2 is drained via the drainage conduit 61, the liquid in the treatment tank 2 is drained without being filtered by the filter 64.

Further, when the filter 64 is inserted in the vertical conduit portion 61a, the filter 64 covers the circulation port 63. In the present embodiment, an inner circumferential surface of the filter 64 having a cylindrical shape is the first surface 64a and an outer circumferential surface of the filter 64 is the second surface 64b.

The filter 64 is held by the filter holding cylinder 67 having a cylindrical shape. The filter holding cylinder 67 is a member for maintaining a shape of the filter 64 having a cylindrical shape. Therefore, when the shape of the filter 64 can be maintained by a rigidity of the filter 64, the filter holding cylinder 67 is unnecessary.

The filter holding cylinder 67 of the present embodiment has a cylindrical shape, both ends of which are opened, and the filter 64 is arranged on the outer circumferential surface or the inner circumferential surface. In the present embodiment that is illustrated, as an example, the filter 64 is sticking to the inner circumferential surface of the filter holding cylinder 67, but the present invention is not limited to the configuration. For example, the filter 64 and the filter holding cylinder 67 may be integrally molded.

The filter holding cylinder 67 can be inserted from the liquid lead-out port 60 into the vertical conduit portion 61a. When the filter holding cylinder 67 is inserted in the vertical conduit portion 61a, an upper end-portion opening 67a provided in the end portion facing upward is opened toward the liquid lead-out port 60. Further, when the filter holding cylinder 67 is inserted in the vertical conduit portion 61a, a lower end-portion opening 67f provided in the end portion facing downward is opened toward the on-off valve 62.

Further, one or a plurality of side-surface portion openings 67b that pass through the filter holding cylinder 67 from outer circumferential surface to the inner circumferential surface are formed in the filter holding cylinder 67. The side-surface portion openings 67b are arranged in positions that overlap with the filter 64. Further, when the filter holding cylinder 67 is inserted in the vertical conduit portion 61a, the side-surface portion openings 67b are arranged in positions that are opened toward the circulation port 63.

Seal members 67c that come into close contact with the inner circumferential surface of the vertical conduit portion 61a are provided above and below a region of the outer circumferential surface of the filter holding cylinder 67 in which the side-surface portion openings 67b are formed. By the pair of seal members 67c, the flow path of the liquid between an inner side of the filter holding cylinder 67 and the circulation port 63 is only limited to routes that pass through the side-surface portion openings 67b. That is, in the present embodiment, all the liquid passing through the filter 64 passes through the side-surface portion openings 67b.

Further, in FIG. 9, the filter holding cylinder 67 is positioned in the vertical conduit portion 61a when a lower end portion 67d abuts against the on-off valve 62 also serving as the holding portion 65. That is, in the present embodiment, a part of the on-off valve 62 exposed to the inside of the vertical conduit portion 61a configures the holding portion 65 that holds the filter 64 in a position that covers the circulation port 63. Further, the filter holding cylinder 67 has a flange shape that is caught in the liquid lead-out port, and hence serves as the holding portion 65. In FIG. 9, the holding portion is formed on both the filter and the drainage conduit, but the holding portion may be formed on one of the filter and the drainage conduit.

As illustrated in FIG. 10, a handle 67e is provided on an upper end portion of the filter holding cylinder 67 so that the filter holding cylinder 67 is easily inserted into and removed from the vertical conduit portion 61a. Further, in the present embodiment, the filter 64 has a cylindrical shape that is inserted in the vertical conduit portion 61a, and hence the filter 64 can be turned about a vertical axis in the vertical conduit portion 61a as illustrated in FIG. 11 when the user operates the handle 67e.

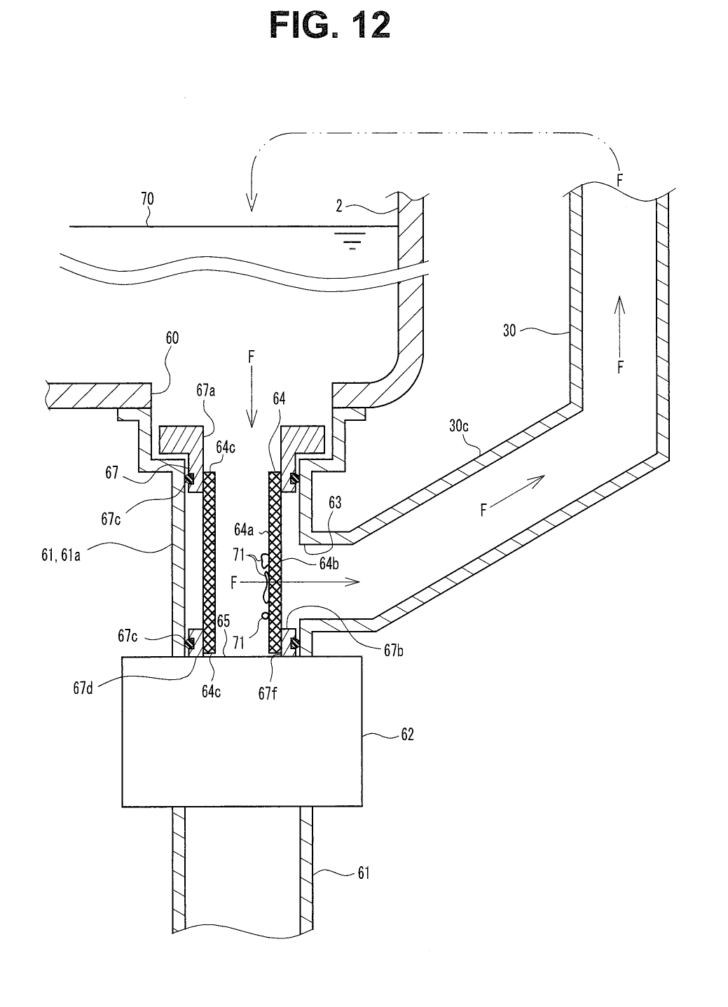

FIG. 12 indicates a flow of the liquid 70 when the circulation operation of the endoscope reprocessor 1 of the present embodiment is executed by arrows F. As illustrated in FIG. 12, during the circulation operation, the liquid 70 accumulated in the treatment tank 2 flows into the vertical conduit portion 61a of the drainage conduit 61 from the liquid lead-out port 60, and flows into a space in the filter 64 from the opening in the end portion 64c facing upward of the filter 64 having a cylindrical shape. Further, the liquid 70 flowing in a space in the filter 64 flows so as to pass through the filter 64 from the first surface 64a that is the inner circumferential surface of the filter 64 toward the second surface 64b that is the outer circumferential surface, and then return into the treatment tank 2 through the circulation port 63 and the circulation conduit 30. Therefore, the foreign matter 71 detached from the endoscope and floating in the liquid 70 is collected by the filter 64 and adheres to the first surface 64a of the filter 64.

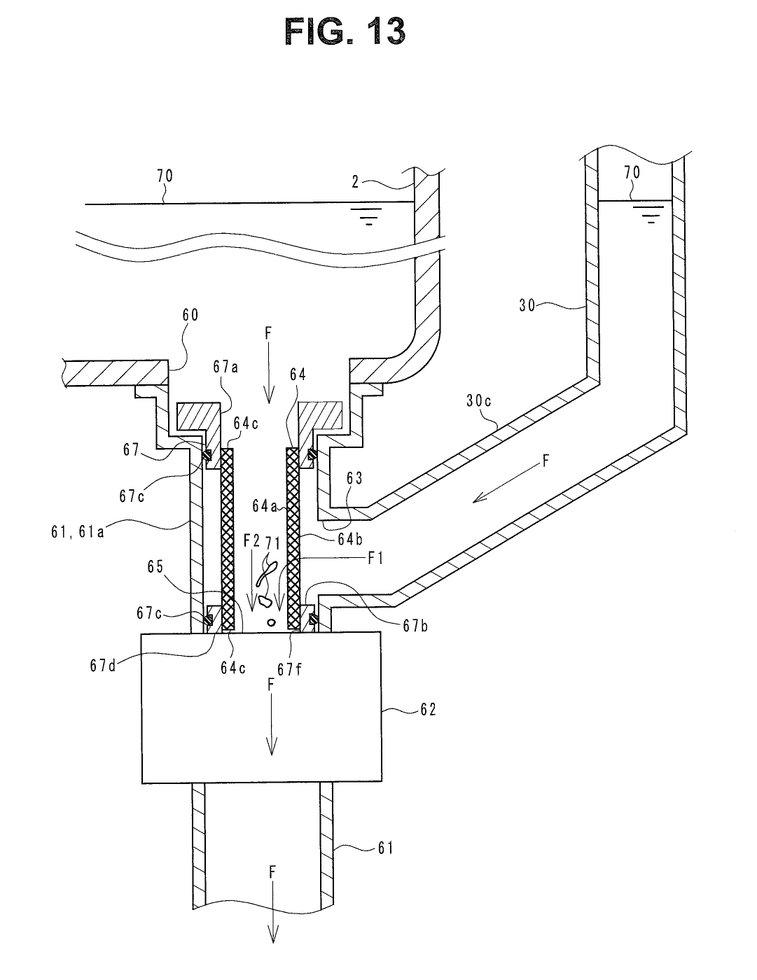

FIG. 13 illustrates a flow of the liquid when the drainage operation of the endoscope reprocessor 1 of the present embodiment is executed. When the drainage operation starts by causing the on-off valve 62 is to be in the open state, the liquid 70 accumulated in the treatment tank 2 is drained through the drainage conduit 61 and the on-off valve 62. Further, when the drainage operation starts, at least a part of the liquid 70 accumulated in the circulation conduit 30 flows toward the circulation port 63, and is drained through the circulation port 63, the filter 64, and the on-off valve 62.

Now, when the flow of the liquid 70 near the filter 64 during the drainage operation is focused on, during the drainage operation, a first flow F1 in which the liquid 70 passes through the filter 64 from the second surface 64b toward the first surface 64a from the inside of the circulation conduit 30 and flows into a space in the filter 64, and a second flow F2 in which the liquid 70 flows into a space in the filter 64 from the inside of the treatment tank 2 and then flows down to the lower side along the first surface 64a of the filter 64 are generated.

As in the first embodiment, the first flow F1 in the liquid 70 generated during the drainage operation applies force to the foreign matter 71 adhering to the first surface 64a of the filter 64 in a direction in which the foreign matter 71 is detached from the first surface 64a into an inner space in the filter 64. Therefore, by the first flow F1 in the liquid 70, the foreign matter 71 adhering to the first surface 64a of the filter 64 is placed in a state that is detached or easily detached from the first surface 64a.

Further, the second flow F2 in the liquid 70 generated during the drainage operation causes the foreign matter 71 to be detached from the first surface 64a by applying shear force to the foreign matter 71 adhering to the first surface 64a of the filter 64. Further, the second flow F2 in the liquid 70 washes away the foreign matter 71 detached from the first surface 64a to the on-off valve 62 and discharges the foreign matter 71 from the inside of the vertical conduit portion 61a.

As described above, the endoscope reprocessor 1 of the present embodiment detaches the foreign matter 71 collected by the filter 64 from the filter 64 and discharges the foreign matter 71 with the liquid 70 when the drainage operation of the liquid 70 accumulated in the treatment tank 2 is executed. That is, the endoscope reprocessor 1 of the present embodiment can automatically remove the foreign matter 71 from the filter 64. As a result, the frequency of cleaning work of the filter 64 manually performed by the user can be reduced, for example.

Further, in the present embodiment, the filter 64 can be turned about the vertical axis in the vertical conduit portion 61a. When the filter 64 has a cylindrical shape as in the present embodiment, regions at which the liquid 70 passes through the filter 64 in the circulation operation tend to be regions close to the circulation port 63. Therefore, in the present embodiment, when the circulation operation is performed, the foreign matter 71 tends to adhere to partial regions of the inner circumferential surface (first surface 64a) of the filter 64 in a circumferential direction about the vertical axis in a concentrated manner.

As described above, the foreign matter 71 mainly adhering to partial regions of the inner circumferential surface of the filter 64 is detached and discharged by performing the drainage operation. However, when the foreign matter is a foreign matter difficult to be detached such as mucus, there is a possibility that a part of the foreign matter 71 remains adhering to the filter 64 even when the drainage operation is performed.

In the present embodiment, by turning the filter 64 about the vertical axis by 90 degrees or 180 degrees, for example, in the vertical conduit portion 61a after executing the reprocessing a predetermined number of times, a region to which the adherence of the foreign matter 71 has been light in the reprocessing executed before can be brought near the circulation port 63.

Therefore, in the present embodiment, even when a state in which the foreign matter 71 remains adhering to the filter 64 occurs, a reduction in a flow rate due to the existence of the foreign matter 71 in the circulation operation performed later can be suppressed or prevented by turning the filter 64. Further, in the abovementioned case, there is a possibility that the foreign matter 71 that remains adhering to regions distant from the circulation port 63 of the filter 64 is detached by the second flow F2 in the liquid 70 in the drainage operation that is performed later. Therefore, in the endoscope reprocessor 1 of the present embodiment, by regularly turning the filter 64, the frequency of cleaning work of the filter 64 manually performed by the user can be reduced, for example.

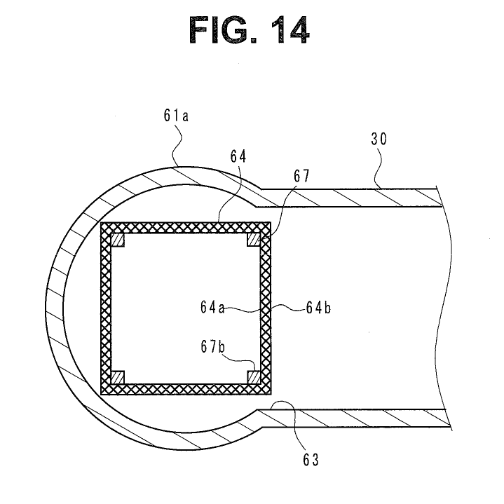

Note that the shape of the filter 64 only needs to be a cylindrical shape, and is not limited to a circularly cylindrical shape. For example, as illustrated in FIG. 14 as a first modification, the filter 64 may have a cylindrical shape, an external form of which is a square when seen from above in a state in which the filter 64 is inserted in the vertical conduit portion 61a. Further, the filter 64 may have a cylindrical shape, an external form of which is a polygon such as a triangle or a hexagon when seen from above in a state in which the filter 64 is inserted in the vertical conduit portion 61a.

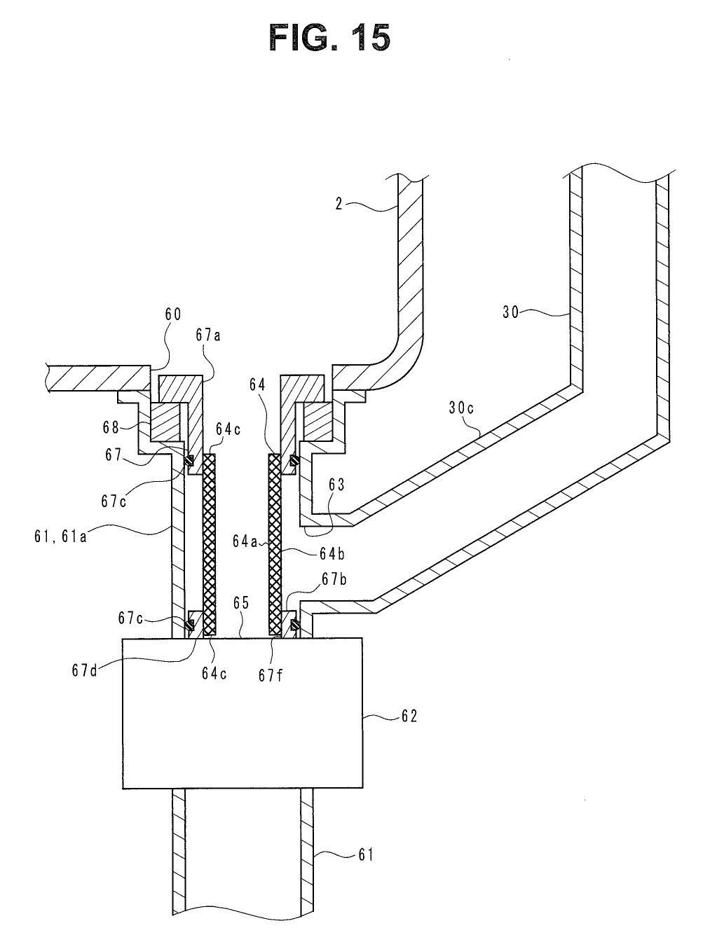

Further, the endoscope reprocessor 1 may have a configuration in which the filter 64 inserted in the vertical conduit portion 61a is turned about the vertical axis. For example, as illustrated in FIG. 15 as a second modification, the endoscope reprocessor 1 may include a ring-shaped ultrasound motor 68 arranged along the inner circumferential surface of the vertical conduit portion 61a, and may have a configuration in which the filter 64 or the filter holding cylinder 67 is turned by the ultrasound motor 68.

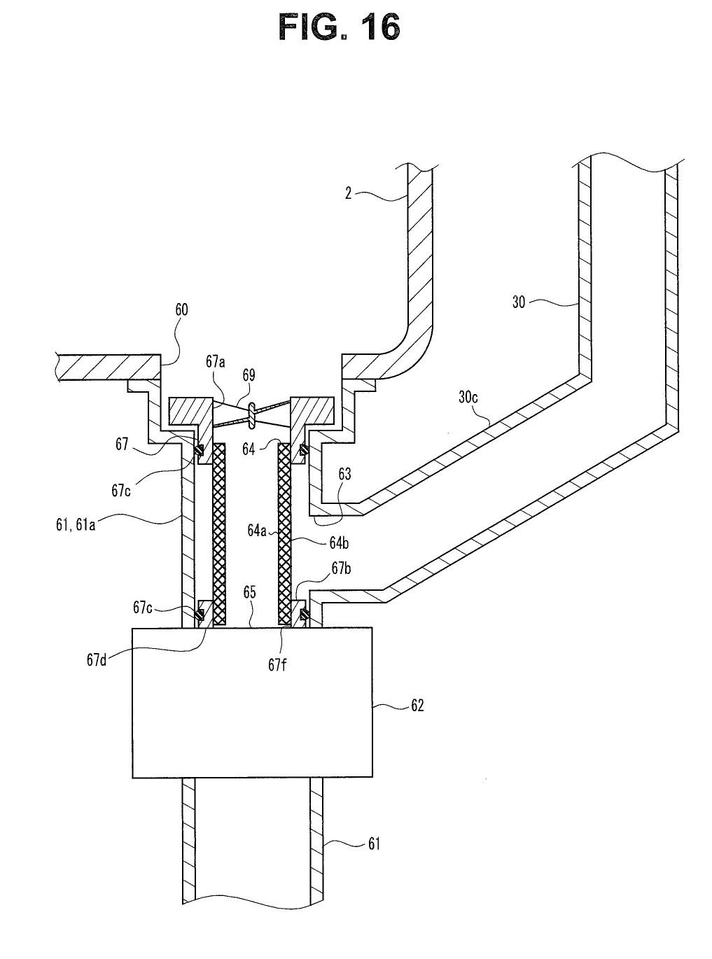

Further, for example, as illustrated in FIG. 16 as a third modification, the filter 64 or the filter holding cylinder 67 may include a water wheel 69 that rotates about the vertical axis in accordance with the flow of the liquid 70 flowing in the vertical conduit portion 61a in the vertical direction. In the modification illustrated in FIG. 16, blades of the water wheel 69 are fixed to the filter holding cylinder 67, and the filter holding cylinder 67 and the filter 64 turn about the vertical axis every time the liquid flows in the vertical conduit portion 61a.

Further, the modifications illustrated in FIG. 5 to FIG. 9 in the first embodiment can be also applied to the endoscope reprocessor 1 of the present embodiment.

### Third Embodiment

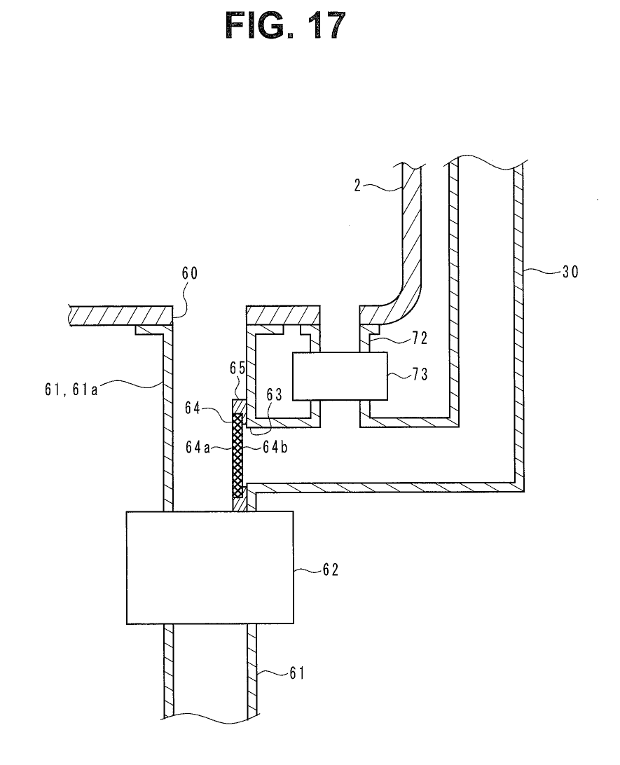

A third embodiment of the present invention is described below. Only differences from the first embodiment are described below, components similar to components in the first embodiment are denoted by the same reference characters, and description of such components is omitted as appropriate. FIG. 17 is a view illustrating the liquid lead-out port 60, the vertical conduit portion 61a, the circulation port 63, the filter 64, and the holding portion 65 of the endoscope reprocessor 1 of the present embodiment in an enlarged manner.

As illustrated in FIG. 17, the endoscope reprocessor 1 of the present embodiment is different from the first embodiment in that the endoscope reprocessor 1 of the present embodiment includes a bypass conduit 72 and a bypass conduit on-off valve 73.

The bypass conduit 72 is a conduit communicating with the inside of the treatment tank 2 and the inside of the circulation conduit 30. Note that the bypass conduit 72 is opened to the bottom surface of the treatment tank 2 in the illustrated embodiment as an example, but the bypass conduit 72 may be opened to a section, which is above the circulation port 63 of the drainage conduit 61 and not covered with the filter 64.

The bypass conduit on-off valve 73 is arranged in the bypass conduit 72. The bypass conduit on-off valve 73 can be switched between a closed state in which the bypass conduit 72 is closed, and an open state in which the bypass conduit 72 is opened. The bypass conduit on-off valve 73 is electrically connected to the control unit 5, and the operation of the on-off valve 62 is controlled by the control unit 5.

The control unit 5 causes the bypass conduit on-off valve 73 to be in the open state only when the drainage operation is executed, and causes the bypass conduit on-off valve 73 to be in a closed state otherwise.

In the endoscope reprocessor 1 of the present embodiment, by causing the bypass conduit on-off valve 73 to be in the open state when the drainage operation is executed, a part of the liquid 70 accumulated in the treatment tank 2 can be caused to flow into the circulation conduit 30 in the drainage operation. That is, in the present embodiment, a part of the liquid 70 accumulated in the treatment tank 2 can be caused to flow so as to pass through the filter 64 from the second surface 64b toward the first surface 64a in the drainage operation.

Therefore, in the endoscope reprocessor 1 of the present embodiment, the volume of the liquid flowing in a direction in which the foreign matter 71 is detached from the filter 64 in the drainage operation can be increased, and hence the foreign matter can be removed from the filter 64 in a more reliable manner.

Note that the modifications illustrated in FIG. 5 to FIG. 9 in the first embodiment can be also applied to the endoscope reprocessor 1 of the present embodiment. Further, the endoscope reprocessor 1 of the present embodiment may include the filter 64 having a cylindrical shape as in the second embodiment. In addition, when the endoscope reprocessor 1 of the present embodiment includes the filter 64 having a cylindrical shape similar to the filter 64 having a cylindrical shape in the second embodiment, the modifications illustrated in FIG. 14 to FIG. 16 in the second embodiment can be applied.

The present invention is not limited to the abovementioned embodiments and can be changed, as appropriate, within a scope not deviated from the spirit or the concept of the present invention that can be read from the claims and the entire specification, and an endoscope reprocessor changed as above is also included in the technical scope of the present invention.

What is claimed is:

1. An endoscope reprocessor, comprising:
a treatment tank in which an endoscope is arranged;
a drainage conduit including a liquid lead-out port opened in a bottom surface of the treatment tank;
an on-off valve arranged in an intermediate position of the drainage conduit;
a circulation conduit including a circulation port, the circulation port being opened between the on-off valve and the liquid lead-out port of the drainage conduit and in a position closer to a ground than the liquid lead-out port, the circulation conduit introducing liquid introduced from the circulation port to the treatment tank;
a filter arranged in the drainage conduit and covering the circulation port; and
a holding portion arranged in the filter or the drainage conduit, the holding portion holding the filter at a position at which the circulation port is covered and liquid that flows into the drainage conduit from an inside of the treatment tank through the circulation port passes through the filter and liquid that flows toward the on-off valve from the inside of the treatment tank through the liquid lead-out port flows along a surface of the filter when the on-off valve is in an open state.

2. The endoscope reprocessor according to claim 1, wherein the drainage conduit includes a vertical conduit portion vertically arranged at least from the liquid lead-out port to the circulation port.

3. The endoscope reprocessor according to claim 1, wherein the circulation conduit includes an inclined conduit portion inclined in a direction away from the ground when the circulation port is a base point.

4. The endoscope reprocessor according to claim 1, wherein the filter includes a cylindrical shape.

5. The endoscope reprocessor according to claim 1, wherein the holding portion is the on-off valve.
